(19) United States
(12) Patent Application Publication
(10) Pub. No.: US 2021/0338708 A1
(43) Pub. Date: Nov. 4, 2021

(54) METHOD FOR TREATMENT OF CORONAVIRUS INFECTION

(71) Applicant: Renibus Therapeutics, Inc., Southlake, TX (US)

(72) Inventors: Donald Jeffrey KEYSER, Southlake, TX (US); Alvaro F. GUILLEM, Lantana, TX (US); Bhupinder SINGH, Phoenix, AZ (US); Richard A. ZAGER, Seattle, WA (US); Stacey RUIZ, Lantana, TX (US)

(21) Appl. No.: 17/205,521

(22) Filed: Mar. 18, 2021

Related U.S. Application Data
(60) Provisional application No. 62/992,083, filed on Mar. 19, 2020.
(60) Provisional application No. 62/706,520, filed on Aug. 21, 2020.

(51) Int. Cl. A61K 31/714; A61K 33/30; A61K 33/24; A61K 47/54; A61K 9/00; A61P 31/14; A01N 25/34; A01N 55/04; A01N 55/02; A61L 9/012

(52) U.S. Cl. CPC A61K 31/714; A61K 33/30; A61K 33/24; A61K 47/546; A61K 9/0043; A61L 2101/44; A01N 25/34; A01N 55/04; A01N 55/02; A61L 9/012; A61P 31/14

(57) ABSTRACT

The present invention involves a novel method for treatment of coronavirus infection, including SARS-COV-2. The method comprises administering stannous protoporphyrin and/or cyanocobalamin to a human patient at risk for developing complications from coronavirus infection. The method is particularly useful where a patient has been diagnosed with coronavirus infection or has been exposed to coronavirus but has not developed symptoms of coronavirus infection.

### CROSS-REFERENCE TO RELATED APPLICATIONS

This application claims the benefit of U.S. Provisional Application No. 62/992,083, filed on Mar. 19, 2020, and U.S. Provisional application No. 62/706,520, filed on Aug. 21, 2020 which applications are hereby incorporated herein by reference.

### BACKGROUND

Since December 2019, the world has experienced an outbreak of coronavirus disease 2019 (COVID-19), a virus-infected pneumonia, caused by severe acute respiratory syndrome coronavirus 2 (SARS-CoV-2). The clinical course of illness, including viral shedding, has not been well described.

Early studies from clinical work originating in Wuhan, China have detailed the clinical course and risk factors for mortality of adult inpatients with COVID-19. See Fei Zhou et al., “Clinical course and risk factors for mortality of adult inpatients with COVID-19 in Wuhan, China: a retrospective cohort study,” The Lancet, Mar. 9, 2020. One particular problem with SARS-CoV-2 is its clinical course progresses after testing positive with milder conditions such as fever and cough through development of sepsis and acute respiratory distress syndrome (ARDS), with the more severe symptoms developing after a week or more from the initial positive test as in FIG. 1.

After severe respiratory symptoms develop, the patient is admitted to the intensive care unit (ICU) and subject to respiratory support which may include non-invasive ventilation, or in large number of cases, intubation and mechanical ventilation. Some patients may also require vasopressor support. Since ARDS tends to be very severe, patients may also require prone ventilation to improve oxygenation. Occasionally patients may need Extra Corporeal Membrane Oxygenation (ECMO) if mechanical ventilation is not sufficient. Despite these measures, the disease carries an overall mortality rate of 3.5%, which is much higher among patients who are admitted to the ICU. It has been noted that cariogenic shock, increased Troponin levels and fatal arrhythmias occur as terminal events in several cases. Mortality following ICU admission is more commonly associated with elderly patients and those suffering from comorbidities such as hypertension, diabetes, coronary heart disease, immunocompromised state, or chronic obstructive lung disease. Attempts have been made to reverse the course of the disease by treating patients in the ICU with anti-viral drugs, such as Remdesivir (Gilead, Inc.) or Favipiravir (Toyama Chemical, Fujifilm group), anti-inflammatory drugs, such as Kevzara (Regeneron) or Actemra (Roche), and the anti-malaria drug chloroquine or hydroxychloroquine (Bayer, Germany).

There remains a need for a treatment that can be safely administered before severe respiratory symptoms develop from a SARS-CoV-2 infection that can reverse the clinical progression of disease resulting from the virus.

### DESCRIPTION OF THE DRAWINGS

FIG. 1: Clinical courses of major symptoms and outcomes and duration of viral shedding from illness onset in patients hospitalized with COVID-19.

FIG. 2: In-vitro antiviral activity of Stannous protoporphyrin (SnPP) on direct SARS-CoV-2 inactivation.

FIG. 3A: In-vitro antiviral activity of SnPP on SARS-CoV-2 replication.

FIG. 3B: In-vitro antiviral activity of Remdesivir on SARS-CoV-2 replication.

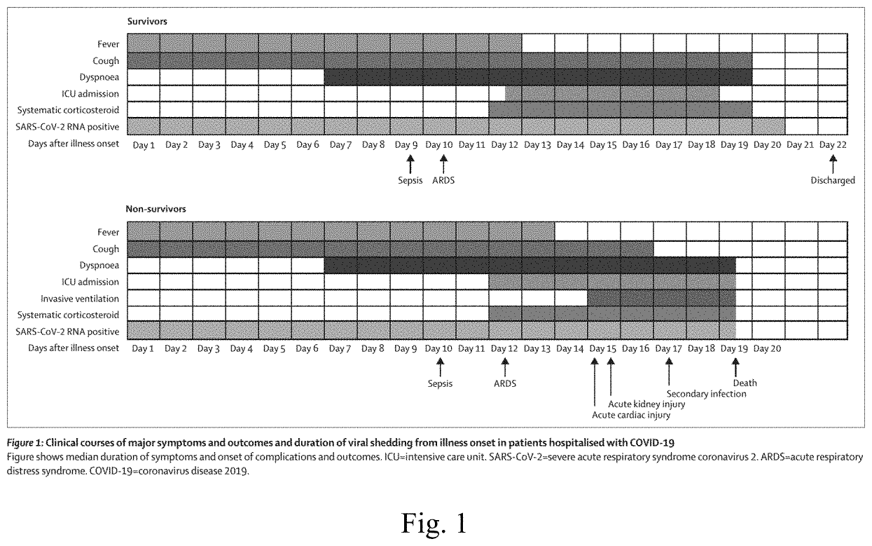

### DETAILED DESCRIPTION

The present inventors' work with protoporphyrin and cyanocobalamin has suggested an early stage treatment of coronavirus infection, particularly from enveloped virus infections that progress slowly after detection such as SARS-CoV-2. The progression of this coronavirus infection provides a clinical window for intervention that provides an opportunity for avoiding or slowing progression of the infection shortly after detection or exposure to the virus. In the case of an outbreak of infection, early intervention is important given the limited availability of intensive care unit (ICU) beds, and high mortality levels after the disease progresses to this stage.

Stannous protoporphyrin (SnPP) has been shown to inhibit viral infection of dengue virus and yellow fever virus by inhibiting synthesis of nonstructural protein 1 (NSP1). Assunção-Miranda et al., “Inactivation of Dengue and Yellow Fever viruses by heme, cobalt-protoporphyrin IX and tin-protoporphyrin IX,’ J. of Applied Microbiol. (2015).

Nonstructural proteins (NSPs) play a role in viral replication, and NSP1 in particular plays a role early in viral in infection, as it is one of the first proteins produced by the virus and is critical for blocking the innate immune response that protects the body from viral infection. In SARS coronavirus, NSP1 is a known virulent factor that has been a target for vaccine development. When engineered without NSP1, SARS coronavirus is weakened and detectable by the immune system, preventing full infection. Narayanan K et al., Virus Res.

NSP1 forms part of the viral replicase transcriptase complex in SARS-CoV-2, the virus that causes COVID-19. Dong S. et al., 2020 J. Med. Virol.; Almeida M S et al., 2007 J. Virol. The present inventors have shown that cyanocobalamin (“CCB”, also known as vitamin B12) can recapitulate the protection seen with SnPP because CCB includes cobalt and induces HO-1 without the temporary HO-1 inhibitory effects. Treatment with SnPP or CCB may be beneficial in the early stages of COVID-19, as it has the potential to: 1) prevent viral replication of SARS-CoV-2 and 2) allow viral detection and clearance by the immune system.

In one embodiment, the invention involves treating an enveloped viral infection, preferably SARS-CoV-2, infection by administering a metal protoporphyrin or metal mesoporphyrin to a human patient, wherein the patient is at risk for developing complications from coronavirus infection. The metal protoporphyrin or metal mesoporphyrin may be one or more of tin protoporphyrin ix (SnPP), tin mesoporphyrin ix, cobalt protoporphyrin ix, cobalt mesoporphyrin ix, zinc protoporphyrin, or zinc mesoporphyrin. Other protoporphyrins may also be used. In one aspect, the protoporphyrin is administered intravenously.

In one aspect, the invention involves treating a coronavirus, preferably SARS-CoV-2, infection by administering a cobalamin to a human patient, wherein the patient is at risk for developing complications from coronavirus infection. The cobalamin may be a cyanocobalamin (i.e., vitamin B12), hydroxocobalamin, methylcobalamin, adenosylcobalamin, or 5-deoxyadenosyl cobalamin. In one aspect, the cobalamin is administered intravenously. In another aspect, the cobalamin is administered orally.

One aspect involves the timing of administration of the methods of treatment. For example, treatment may occur at the time a patient tests positive for the coronavirus infection. In one respect, the patient may have tested positive for the coronavirus infections and not developed symptoms of pneumonia or ARDS. In another aspect, the patient may have been exposed to a coronavirus infection. Where a patient exhibits one or more risk factors associated with the coronavirus infection, it may be more important to administer the treatment without a positive test for the virus.

For example with respect to SARS-CoV-2, the patient's risk factors include being of age 60 or above, having a d-dimer level of greater than 1 μg/L, greater than 1.5 μg/L, greater than 2.0 μg/L, or between 1 and 50 μg/L, or between 5 and 50 μg/L, having a structural organ failure assessment (SOFA) score of 1 or above, greater than 2, or between 2 and 10, or in the range of 2.5 to 6. In another aspect, the patient may be suffering from a comorbidity such as hypertension, diabetes, coronary heart disease, and/or chronic obstructive lung disease.

The treatment in accordance with the invention may be administered a single time. Preferably the dose is administered followed by another test for the coronavirus. If the patient continues to test positive for the coronavirus, the patient is then administered another treatment in accordance with the present invention. In one aspect, the cycle of treatment followed by testing is repeated until the patient no longer tests positive, or until symptoms develop to the point where ICU stabilization is necessary. In one aspect, the second test for coronavirus takes place 12 hours after the treatment. Other treatment intervals may be desirable including 24 hours, 36 hours, 48 hours or 72 hours or up to a week after the previous treatment.

In one aspect, the administered dose of protoporphyrin or cobalamin is effective for lowering or eliminating the viral load of the patient. This may include doses within the range of, for example, 0.5 to 2.5 mg/kg of protoporphyrin, preferably 0.6 to 2.15 mg/kg, more preferably 1 to 1.6 mg/kg. In one aspect, the dose ranges from 45 to 150 mg, preferably 50 to 120 mg, for example about 90 mg.

In one aspect, the protoporphyrin or cobalamin, particularly SnPP, may be administered intranasally. The intranasal composition may be particularly effective in a setting where an enveloped virus such as a respiratory virus is known to spread rapidly, such as a nursing home, jail, meatpacking facility. The patient receiving the treatment may be one who has been exposed and could range to a patient experiencing a cytokine storm as a result of the patient's immune response to the viral infection, as has been known to occur in some patients infected with SARS-COV-2. The intranasal composition may be administered as a lavage, drop, squirt system, spray, nasal screen, cleaning solution, or swab. The intranasal composition may be a topical liquid, a topical gel, or an inhaled solution, which may have a physiologically compatible pH. For example, a pH between 5.5-6.5. The solution may also be isotonic to reduce discomfort at the time of intranasal administration.

In another aspect, the administration occurs by applying the anti-viral composition to a facemask. Specifically, upon applying to the facemask the metal protoporphyrin (e.g., SnPP) or metal mesoporphyrin can act upon the patient or further reduce the spread of infection. In one aspect, upon applying to the facemask the metal protoporphyrin or metal mesoporphyrin enters the nasal cavity of the person in an amount effective to treat or prevent the enveloped virus infection. In another aspect, particles of the enveloped virus exhaled or expelled by the person into the facemask are destroyed or neutralized by the antiviral composition. Both actions may occur simultaneously or sequentially, and may reduce community spread of the virus and confer additional protection upon those wearing the mask that improves the effectiveness of the mask. The term mask includes other articles that are placed over the face in a manner similar to a surgical or N95 mask, such as a bandana, gaiter, knit mask.

### Example 1

A patient having tested positive for SARS-CoV-2 infection is administered at the time of the positive test a dose of stannous protoporphyrin ix. The patient is given a single dose of 90 mg intravenous stannous protoporphyrin ix. The patient is then again tested for SARS-CoV-2 three days later.

### Example 2

A patient having tested positive for SARS-CoV-2 infection is intravenously administered at the time of the positive test a dose of cyanocobalamin (1 mg/mL). The administered dose is 1 mg. The patient is then again tested for SARS-CoV-2 three days later

### Example 3

A Phase 2, randomized, placebo-controlled study to evaluate the effect of SnPP and CCB on preventing progression of asymptomatic or mildly symptomatic COVID-19 in high risk individuals using Stannous protoporphyrin (SnPP) or Cyanocobalamin (CCB).

The objective of the study is to evaluate the severity of COVID-19 through Day 14 using the 7-point ordinal scale. The ordinal scale is an assessment of the clinical status at the first assessment of a given study day. The scale is as follows:

1) Death;

2) Hospitalized, on invasive mechanical ventilation or extracorporeal membrane oxygenation (ECMO);

3) Hospitalized, on non-invasive ventilation or high flow oxygen devices;

4) Hospitalized, requiring supplemental oxygen;

5) Hospitalized, not requiring supplemental oxygen;

6) Not hospitalized, limitation on activities;

7) Not hospitalized, no limitations on activities.

Secondary objectives include assessing the following parameters through 28 days after dosing: Viral titer, Fever incidence, Change in Patient Reported Outcomes Measurement Information System (PROMIS) dyspnea functional limitations and severity, Length of hospital stay, Oxygen-free days, Percentage of subjects progressing to ICU, Days on ventilator, Mortality, and/or Safety.

Approximately 150 subjects are planned to be enrolled and will be randomized 1:1:1 to receive SnPP 90 mg, CCB XX mg, or placebo (normal saline). The study duration is 30 days per subject.

SnPP will be administered as a single dose of 90 mg via intravenous (IV) infusion over a 180-minute period on study Day 1. CCB will be administered as single dose via intramuscular (IM)/IV over a 180-minute period on study Day 1/daily for X days. Placebo will be administered via IV over a 180-period on study Day 1.

Inclusion Criteria:

1. Male or female subjects age>18 years at Screening.

2. Documented infection with SARS-CoV-2 within the preceding 7 days.

3. Asymptomatic or exhibiting mild symptoms of COVID-19, with mild symptoms defined as:
Fever
Mild cough
Body aches
Sore throat
Headache

4. Gastrointestinal symptoms due to COVID-19

5. Subjects who have a high risk of disease progression within 1-2 weeks (age>60 years or comorbidities including diabetes, immunocompromised state, advanced chronic kidney disease, active malignancy, organ transplant status).

6. Subjects who are admitted to a hospital or a controlled facility for observation and standard of care treatment.

7. Subjects who are not requiring supplemental oxygen.

8. If female, must be post-menopausal, surgically sterile, or in case of female subjects with child-bearing potential, must be practicing two effective methods of birth control during the study and through 28 days after completion of the study.

9. For females with child-bearing potential, a pregnancy test must be negative at the Screening Visit.

10. If male, must be surgically sterile or willing to practice two effective methods of birth control during the study and through 30 days after completion of the study.

Must be willing and able to give informed consent and comply with all study procedures.

Exclusion criteria:

1. Immediate need or anticipated need for ICU care and/or ventilator support.

2. Known or suspected sepsis at time of Screening.

3. Pregnancy or lactation.

4. History of photosensitivity or active skin disease that, in the opinion of the investigator, could be worsened by SnPP.

5. Known hypersensitivity or previous anaphylaxis to SnPP or any tin-based product.

6. Treatment with an investigational drug or participation in an interventional trial within 30 days prior to the first dose of study drug.

7. Inability to comply with the requirements of the study protocol.

Study Design:

This is a Phase 2, multicenter, randomized, placebo-controlled study to evaluate the effect of SnPP on preventing the progression of asymptomatic/mildly symptomatic COVID-19 in individuals who have a high risk of disease progression. Subjects will be randomized 1:1:1 to receive a single dose of SnPP, CCB, or placebo via IV infusion within 7 days after diagnosis of COVID-19.

The study is designed as follows:
A screening period with baseline evaluations for study eligibility.
If subjects meet the eligibility criteria, they will receive SnPP, CCB, or placebo via IV infusion over a 180-minute period.
Subjects will be evaluated through Day 14 for:
Severity of COVID-19 (Ordinal Clinical Scale)
Hospitalization status
Subjects will be evaluated for the following through 28 days after dosing:
Viral titers of COVID-19
Fever incidence
Change in PROMIS dyspnea functional limitations and severity
Length of hospital stay
Oxygen free days
Progression to ICU
Days on ventilator
Mortality at Day 28
Safety

Efficacy Assessment:

Efficacy assessments through Day 14 include:
Severity of COVID-19 (Ordinal Clinical Scale)
Hospitalization status

Efficacy assessments through Day 28 include:
Viral titers of COVID-19
Fever incidence
Change in PROMIS dyspnea functional limitations and severity
Length of hospital stay
Oxygen free days
Progression to ICU
Days on ventilator
Mortality at Day 28

Safety Assessment
Treatment-emergent adverse events (TEAEs), including incidence, severity, and relationship to study drug
Withdrawals due to adverse events (AEs)
Physical examination findings
Changes in vital signs
Changes in laboratory tests
Concomitant medication use due to AEs

### Example 4—Plaque Assay to Determine the Virus Titer

Approximately, 24 hours prior to the assay, 6-well plates were used and seeded 4×10⁵ Vero E6 cells/well (in 2 ml, Dulbecco's Modified Eagle Medium (DMEM)+10% Fetal Bovine Serum (FBS)+1% P/S). For each sample to be analyzed, 10-fold serial dilutions in DMEM+1% Penicillin, Streptomycin, Glutamine P/S/G were made by pipetting up and down several times. As negative control DMEM+1% P/S/G was used alone. For each compound, 400 μl/well of each serial dilution on the Vero E6 cells was plated, including the negative control. Samples were incubated for 1 h at 37° C., and rocked manually every 15 min. After 1 h, samples were removed and 3 ml of warmed overlay DMEM/F-12/Agar mixture (DMEM-F12 with 1% DEAE-Dextran, 2% agar, and 5% NaHCO₃ containing 2% Agar) was added to each well. Plates were incubated for 3 days at 37° C. with 5% CO₂. After this incubation period, the overlay was removed and the sample was fixed overnight in plate using 10% neutral buffered formalin, followed by one PBS wash. Cells were permeabilized with 0.5% Triton-X-100 for 15-20 minutes at room temperature. Washed three times with phosphate buffered saline (PBS), and then mouse anti-NP SARS-CoV-1/SARS-CoV-2, 1C7C7 antibody at 1 μg/ml in PBS+1% Bovine serum albumin (BSA) was added for 1 h at 37° C., 5% CO₂. After this incubation period, cells were washed with PBS and prepared and biotinylated secondary antibody (150 μl) in 10 ml of PBS containing 150 μl of normal blocking was added for 30 minutes at 37° C., 5% CO₂. (See: Vector laboratories, cat: PK-4000). While plates were incubating, ABC reagent was prepared by adding 2 drops (100ul) of reagent A, and 2 drops (100 μl) of reagent B into 10 ml of PBS. (See: Vector laboratories, cat: PK-4000).

Post-secondary antibody incubation, avidin-biotin complex (ABC) reagent was added and incubated for 30 minutes at room temperature. Plates were washed 3 times with PBS and the substrate solution was prepared by adding the mixture of reagents below in 5 ml of distilled water. Where there was a need to add more substrate, reagents were added proportionately.

2 drops (84 μl) of 3,3′ Diaminobenzidine (DAB) reagent 1

4 drops of (100 μl) of DAB reagent 2

2 drops of (80 μl) of DAB reagent 3

2 drops of (80 μl) of DAB reagent 4

The plates were developed adding Vector DAB substrate as per the manufacturer's instruction (Vector DAB SK-4000). The plates were washed 3 times and plaques were counted and the virus titer calculated using the formula given below:

Virus(PFU/ml)=number of plaques×dilution at which plaques are being counted×1/virus inoculum used in ml.

### Example 5—Cell Culture and Virus Propagation

Vero E6 cells (ATCC #CRL 1586) were cultured in DMEM containing 5-10% FBS and 1% P/S. Cells were maintained at 37° C. and 5% CO₂. Samples of SARS-CoV-2 were obtained from BEI Resources (2019-nCoV/USA-WA1/2020 strain). Stocks were prepared by infection of confluent monolayers of Vero E6 cells (T75 flasks) for two-three days, until a complete cytopathic effect (CPE) was visible. Media were collected and clarified by centrifugation prior to being aliquoted for storage at −80° C. Titer of stock was determined by plaque assay using Vero E6 cells in 6-well plates as described in Example 4. All work with infectious virus is performed in a Biosafety Level 3 laboratory.

Plaque assay for virus quantitation. Culture supernatant from EC₅₀ assays at 24 hrs were collected to quantitate virus titer. EC₅₀ and two adjacent concentrations of SnPP were used to perform traditional Plaque assay in 6- or 12-well culture plate. Cells were observed for the cytopathic effect for 2-3 days and were then fixed and stained and plaques counts were used to calculate the virus titer.

### Example 6—Condition 1. Effective Concentration Determination (EC₅₀) of SnPP. (SARS-CoV-2 Exposure and SnPP Treatment Concurrently)

Vero E6 cells (5×10⁴ cells/well, 96 well plate format, quadruplicates) were seeded. 24 h later, serially diluted SnPP (100 uM-5 nM) was incubated with SARS-CoV-2 (MOI 0.05) at 37° C. for 1 hr. Vero E6 cells (5×10⁴ cells/well, 96 well plate format, quadruplicates) were treated with serially diluted SnPP (100 uM-5 nM) pre-incubated with SARS-CoV-2 (MOI 0.05) for 1 h 37° C. After incubation of cells with the virus-drug mixture for 1 h at 37° C., the virus-drug mixture was removed and post-infection media (DMEM 2% FBS, 1% PS) and avicel was added. Cells were exposed to the virus (no drug), cells only, and cells with vehicle control served as assay controls. At 24 h post-infection, cells were fixed and stained with an anti-NP rabbit polyclonal antibody against SARS-CoV-1 that cross-reacts with SARS-CoV-2. Viral plaques were quantified in an ELISPOT reader.

50 mM stock solution of SnPP was made in dimethyl sulfoxide (DMSO) and aliquots were frozen in −20° C. and a new aliquot was taken out for each experiment. Three-fold serially diluted SnPP (100 μM to 5 nM; three-fold serial dilutions) was incubated with SARS-CoV-2 [Multiplicity of infection (MOI) 0.01] at 37° C.; 5% CO₂ for 1 h. Vero E6 cells (5×10⁴/well; in 96-well culture plate; seeded 24 h before) were infected with the mixture of serially diluted SnPP (100 μM to 5 nM) and SARS-CoV-2 (MOI 0.01) 37° C.; 5% CO₂ for 1 h. After 1 h at 37° C., virus-drug mixture was removed and 1% Avicel containing overlay (DMEM 2% FBS, 1% penicillin-streptomycin-glutamine (PSG)) and was added. Cells exposed to the virus (no drug), cells alone, and cells with vehicle control (DMSO) were included as controls.

At 24 h post-infection, culture supernatants were collected and stored at −80° C. for virus quantitation by the 6-well plaque assay as described in Example 4. Plate was fixed overnight using 10% neutral buffered formalin, followed by 3 times washes with PBS. Cells were permeabilized with 0.5% Triton-X-100 for 15-20 minutes at room temperature. After washing, cells were stained with mouse anti-NP SARS-CoV-2, 1C7C7 antibody at 1 μg/ml in PBS+1% BSA for 1 h at 370 C. After this incubation, cells were washed with PBS and biotinylated secondary antibody (150 μl) in 10 ml of PBS containing 150 μl normal blocking serum was added for 30 minutes at 370 C (See: Vector laboratories, Cat PK-4000). While plate was in incubation, ABC reagent was prepared by adding 2 drops (100 μl) of reagent A, 2 drops (100 μl) of reagent B in 10 ml of PBS (See: Vector laboratories, Cat: PK-4000). Post-secondary antibody incubation, ABC reagent was added for 30 minutes at room temperature. After 3 times washing with PBS, substrate solution was prepared by adding the mixture of reagents below in 5 ml of distilled water and plaques were developed.

2 drops (84 μl) of DAB reagent 1

4 drops of (100 μl) of DAB reagent 2

2 drops of (80 μl) of DAB reagent 3

2 drops of (80 μl) of DAB reagent 4

Plates were read using a CTL ImmunoSpot plate reader and counting software (Cellular Technology Limited, Cleveland, Ohio, USA). Percent virus inhibition, was calculated as follows:

[(Number of plaques from treated condition−plaques from cells only(negative control)/number of plaques from virus only(positive control)]×100.

A non-linear regression curve fit analysis over the dilution curve was performed using GraphPad Prism vs. 7.0 to calculate effective concentration 50 (EC₅₀) of SnPP.

### Results: Condition 1. (Virus Exposure and Drug Treatment Concurrently); Effective Concentration Determination (EC₅₀) of SnPP

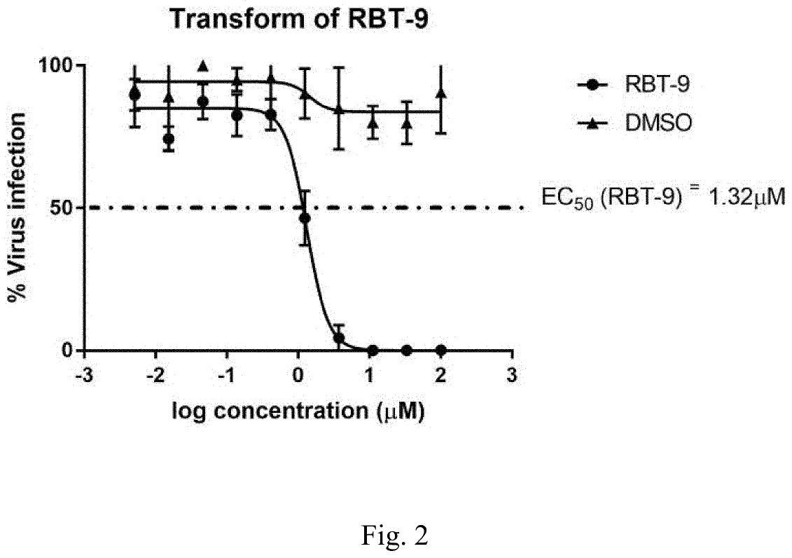

The ability of SnPP (RBT-9) to directly inactivate SARS-CoV-2 was evaluated. SnPP demonstrated anti-viral activity against SARS-CoV-2, with a mean EC₅₀ of 1.32 μM (FIG. 2 and Table 1). A negative vehicle control (DMSO) was also diluted equivalent to SnPP concentration and was found inactive up to its highest concentration. The mean number of plaques obtained in virus infection alone (control) was considered as 100% of virus infection. (Actual numbers have been provided in Table 1)

TABLE 1
SnPP (RBT-9) in vitro antiviral activity on direct SARS-CoV-2
inactivation (Plaque counts)

| | | | | | | | | | | | Virus | Cells |
|---|---|---|---|---|---|---|---|---|---|---|---|---|
| | | | | | | | | | | | (positive | (negative |
| Concentration in (μM) | 100 | 33.33 | 11.11 | 3.70 | 1.23 | 0.41 | 0.14 | 0.046 | 0.015 | 0.005 | control) | control) |

Plaque counts

| | | | | | | | | | | | | | |
|---|---|---|---|---|---|---|---|---|---|---|---|---|---|
| RBT-9 | Replicate 1 | 0 | 0 | 0 | 0 | 189 | 235 | 174 | 196 | well error | 236 | 296 | 0 |
| | Replicate 2 | 0 | 0 | 0 | 0 | 117 | 259 | 217 | 222 | 189 | 203 | 257 | 0 |
| | Replicate 3 | 0 | 0 | 0 | 48 | 64 | 194 | 354 | 332 | well error | 304 | 261 | 0 |
| | Replicate 4 | 0 | 0 | 0 | 0 | 132 | 205 | 230 | 256 | 212 | 260 | 266 | 0 |
| DMSO | Replicate 1 | 318 | 197 | 207 | 298 | 232 | 235 | 267 | 289 | 377 | 285 | 296 | 0 |
| | Replicate 2 | 264 | 237 | 207 | 193 | 228 | 326 | 245 | 309 | 307 | 206 | 257 | 0 |
| | Replicate 3 | 200 | 213 | 234 | 225 | 290 | 305 | 258 | 324 | 182 | 269 | 261 | 0 |
| | | | | | | | | | | | | 266 | 0 |

Virus quantitation from the closest well (1.23 μM well) to the SnPP EC₅₀ concentration (1.32 μM well) was measured showing a 46.2% reduction in viral growth relative to the virus alone control well (0.98×10⁴ PFU/ml vs. 1.83×10⁴ PFU/ml, respectively, Table 2). About 98.6% virus reduction was detected at the concentration of 3.7 μM for SnPP (3-fold higher than SnPP EC₅₀ value). However, no reduction in virus load was detected at the concentration of 0.411 μM for SnPP (3-fold lower than SnPP EC₅₀ value).

TABLE 2
SnPP (RBT-9) in vitro antiviral activity on
direct SARS-CoV-2 inactivation (PFU/ml)

| | | | | | % reduction |
|---|---|---|---|---|---|
| | Rep-1 | Rep-2 | Rep-3 | Average | relative to |
| Sample | (PFU/ml) | (PFU/ml) | (PFU/ml) | (PFU/ml) | virus control |
| Virus only | 2 × 10⁴ | 1.75 × 10⁴ | 1.75 × 10⁴ | 1.83 × 10⁴ | N.A. |
| RBT-9 EC 50 (1.32 μM) | 0.75 × 10⁴ | 1.2 × 10⁴ | 1.0 × 10⁴ | 0.98 × 10⁴ | 46.2 |
| RBT-9 (3.7 μM) | 0.75 × 10⁴ | 0 | 0 | 0.25 × 10⁴ | 98.6 |
| RBT-9 (0.411 μM) | 2.25 × 10⁴ | 1.75 × 10⁴ | 1.5 × 10⁴ | 1.83 × 10⁴ | No reduction |

### Example 7—Condition 2. Effective Concentration Determination (EC₅₀) of SnPP (Drug Treatment after Virus Exposure)

Vero E6 cells (5×10⁴ cells/well, 96 well plate format, quadruplicates) were infected with SARS-CoV-2 (MOI 0.05). After 1 h of viral absorption at 37° C., virus inoculum was removed and post-infection media (DMEM 2% FBS, 1% PS) containing the serially diluted SnPP (100 μM-5 nM) and avicel was added. Cells exposed to virus (no drug), cells only, cells with vehicle and positive control (Remdesivir) were used as internal control. At 24 h post-infection, cells were fixed, permeabilized and stained with an anti-NP rabbit polyclonal antibody against SARS-CoV-1 that cross-react with SARS-CoV-2. Viral plaques were quantified in an ELISPOT reader.

50 mM stock SnPP was made in DMSO and aliquots were frozen in −20° C. and a new aliquot was taken out for each experiment. Vero E6 cells (5×10⁴/well; in 96-well culture plate; seeded 24 h before) were infected SARS-CoV-2 (MOI 0.01) at 37° C.; 5% CO₂ for 1 h. After 1 h of viral absorption at 37° C., 5% CO₂; virus inoculum was removed and post-infection media (DMEM 2% FBS, 1% PS) containing three-fold serially diluted SnPP (100 μM to 5 nM; three-fold serial dilutions) and 1% Avicel was added. Cells exposed to virus (no drug), cells alone, cells with vehicle (DMSO) and control Remdesivir (50 μM-2.5 nM; three-fold serial dilutions) were included as control. At 24 h post-infection, culture supernatants were collected and stored at −80° C. for virus quantitation by the 6-well plaque assay as described in Example 4.

Plates were fixed overnight using 10% neutral buffered formalin, followed by 3 times washes with PBS. Cells were permeabilized with 0.5% Triton-X-100 for 15-20 minutes at room temperature. After washing, cells were stained with mouse anti-NP SARS-CoV-2, 1C7C7 antibody at 1 μg/ml in PBS+1% BSA for 1 h at 37° C. After this incubation, cells were washed with PBS and biotinylated secondary antibody (150 μl) in 10 ml of PBS containing 150 μl normal blocking serum was added for 30 minutes at 370 C (See: Vector laboratories, Cat PK-4000). While plates were incubating, ABC reagent was prepared by adding 2 drops (100 μl) of reagent A, and 2 drops (100 μl) of reagent B to 10 ml of PBS (See: Vector laboratories, Cat: PK-4000). Post-secondary antibody incubation, ABC reagent was added for 30 minutes at room temperature.

After washing the plate, 3 times with PBS, substrate solution was prepared by adding the mixture of reagents below in 5 ml of distilled water and plaques were developed.

2 drops (84 μl) of DAB reagent 1

4 drops of (100 μl) of DAB reagent 2

2 drops of (80 μl) of DAB reagent 3

2 drops of (80 μl) of DAB reagent 4

Plates were read using a CTL ImmunoSpot plate reader and counting software (Cellular Technology Limited, Cleveland, Ohio, USA). Percent virus inhibition, was calculated as follows:

[(Number of plaques from treated condition−plaques from cells only(negative control)/number of plaques from virus only(positive control)]×100.

A non-linear regression curve fit over the dilution curve was performed using GraphPad Prism vs. 7.0 to calculate the effective concentration 50 (EC₅₀) of SnPP.

### Results

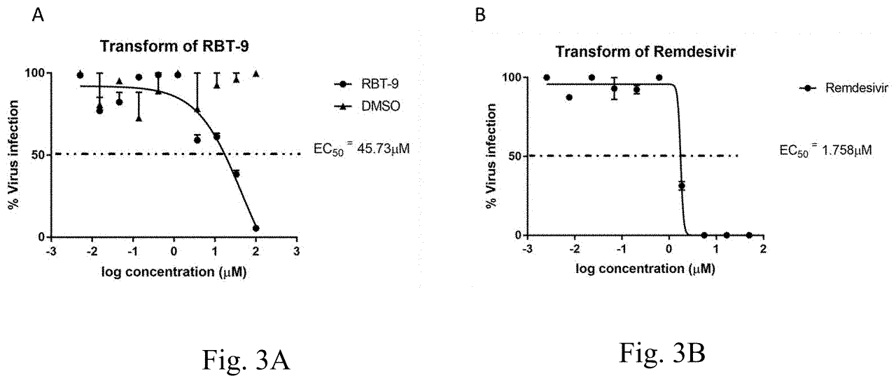

The ability of SnPP to affect SARS-CoV-2 replication was tested. SnPP demonstrated antiviral activity against SARS-CoV-2, with a mean EC₅₀ of 45.73 μM (FIG. 3A and Table 3). Remdesivir served as a positive control (EC₅₀ 1.758 μM; FIG. 3(B) and Table 3). The mean of number of plaques obtained in virus only (control) was considered to represent 100% of virus infection. (Actual numbers have been provided in Table 3).

TABLE 3
In-vitro antiviral activity of SnPP (RBT-9) on viral replication
(Plaque counts)

| | | | | | | | | | | | Virus | Cells |
|---|---|---|---|---|---|---|---|---|---|---|---|---|
| | | | | | | | | | | | (positive | (negative |
| Concentration in (μM) | 100 | 33.33 | 11.11 | 3.70 | 1.23 | 0.41 | 0.14 | 0.046 | 0.015 | 0.005 | control) | control) |

Plaque counts

| RBT-9 | Replicate 1 | 7 | 79 | 145 | 153 | 294 | 304 | 211 | 259 | 164 | 294 | 214 | 0 |
|---|---|---|---|---|---|---|---|---|---|---|---|---|---|
| | Replicate 2 | 15 | 89 | 143 | 130 | 290 | 292 | 293 | 170 | 136 | 363 | 232 | 0 |
| | Replicate 3 | 15 | 99 | 134 | 125 | 286 | 243 | 228 | 167 | 228 | 342 | 219 | 0 |
| | Replicate 4 | 12 | 78 | 129 | 125 | 320 | 309 | 264 | 182 | 172 | 223 | 227 | 0 |
| DMSO | Replicate 1 | 265 | 223 | 190 | 126 | 321 | 235 | 197 | 210 | 137 | 260 | 214 | 0 |
| | Replicate 2 | 222 | 206 | 227 | 265 | 307 | 174 | 127 | 215 | 281 | 369 | 232 | 0 |
| | | | | | | | | | | | | 219 | |
| | | | | | | | | | | | | 227 | |
| | | | | | | | | | | | | Virus | Cells |
| | | | | | | | | | | | | (positive | (negative |
| Concentration in (μM) | | 50 | 16.67 | 5.56 | 1.85 | 0.62 | 0.21 | 0.07 | 0.023 | 0.008 | 0.003 | control) | control) |
| Remdesivir | Replicate 1 | 0 | 0 | 0 | 64 | 274 | 212 | 233 | well error | well error | 353 | 214 | 0 |
| | Replicate 2 | 0 | 0 | 0 | 76 | 270 | 200 | 192 | 245 | 195 | 324 | 232 | 0 |
| | | | | | | | | | | | | 219 | 0 |
| | | | | | | | | | | | | 227 | 0 |

Viral titers were quantitated in neighboring wells (33.33 μM well) surrounding the SnPP EC₅₀ concentration (EC₅₀ of 45.73 μM) by plaque assay. Virus could not be detected at concentrations 3-fold higher than the EC₅₀ (100 μM). However, at concentrations 3-fold lower than the EC₅₀ (11.1 μM) about 48% of virus inhibition was observed relative to control well (virus alone) (2×10⁴ PFU/ml vs. 3.91×10⁴ PFU/ml, respectively).

TABLE 4
In-vitro antiviral activity of SnPP (RBT-9) in at viral replication (PFU/ml)

| | | | | | % reduction |
|---|---|---|---|---|---|
| | Rep-1 | Rep-2 | Rep-3 | Average | relative to |
| Sample | (pfu/ml) | (pfu/ml) | (pfu/ml) | (pfu/ml) | virus control |
| Virus only | 4.5 × 10⁴ | 3.75 × 10⁴ | 3.5 × 10⁴ | 3.91 × 10⁴ | N.A. |
| RBT-9 EC 50 (33.33 μM) | not detected | not detected | not detected | N.A. | |
| RBT-9 (100 μM) | not detected | not detected | not detected | N.A. | |
| RBT-9 (11.11 μM) | 2.25 × 10⁴ | 1.75 × 10⁴ | 2 × 10⁴ | 2 × 10⁴ | 48 |

Results indicate that SnPP shows an antiviral efficacy on direct SARS-CoV-2 inactivation at an EC₅₀ of 1.32 μM and on SARS-CoV-2 replication at an EC₅₀ 45.73 μM as calculated by non-linear regression curve fit analysis GraphPad Prism vs. 7.

Data previously obtained from National Center for Advanced Translational Science (NCATS) group at National Institute of Health (NIH) that stated the CC₅₀ was >30 μM in the Vero E6 cell line in the presence of SARS-CoV-2. The studies in the Examples above did not observe a cytopathic effect (CPE) of SnPP, meaning there was no destruction of the cell (e.g., cell viability was maintained). Thus, the present inventors consider that these data show antiviral efficacy by SARS-CoV-2 inactivation.

### Example 8—Primary CPE Assay

The following assay is described in “Reduction of virus-induced cytopathic effect (Primary CPE Assay), Institute for Antiviral Research, Utah State University, Apr. 28, 2020.

Confluent or near-confluent cell culture monolayers of Vero 76 cells are prepared in 96-well disposable microplates the day before testing. Cells are maintained in minimum essential medium (MEM) supplemented with 5% FBS. For antiviral assays the same medium is used but with FBS reduced to 2% and supplemented with 50-μg/ml gentamicin. Compounds are dissolved in DMSO, saline or the diluent requested by the submitter. Less soluble compounds are vortexed, heated, and sonicated, and if they still do not go into solution are tested as colloidal suspensions. The test compound is prepared at four serial log 10 concentrations, usually 0.1, 1.0, 10, and 100 μl or μM (per sponsor preference). Lower concentrations are used when insufficient compound is supplied. Five microwells are used per dilution: three for infected cultures and two for uninfected toxicity cultures. Controls for the experiment consist of six microwells that are infected and not treated (virus controls) and six that are untreated and uninfected (cell controls) on every plate. A known active drug is tested in parallel as a positive control drug using the same method as is applied for test compounds. The positive control is tested with every test run.

Growth media is removed from the cells and the test compound is applied in 0.1 ml volume to wells at 2× concentration. Virus, normally at −60 CCID 50 (50% cell culture infectious dose) in 0.1 ml volume is added to the wells designated for virus infection. Medium devoid of virus is placed in toxicity control wells and cell control wells. Plates are incubated at 37° C. with 5% CO₂ until marked CPE (>80% CPE for most virus strains) is observed in virus control wells. The plates are then stained with 0.011% neutral red for approximately two hours at 37° C. in a 5% CO₂ incubator. The neutral red medium is removed by complete aspiration, and the cells may be rinsed 1× with phosphate buffered solution (PBS) to remove residual dye. The PBS is completely removed, and the incorporated neutral red is eluted with 50% Sorensen's citrate buffer/50% ethanol for at least 30 minutes. Neutral red dye penetrates into living cells, thus, the more intense the red color, the larger the number of viable cells present in the wells. The dye content in each well is quantified using a spectrophotometer at 540 nm wavelength. The dye content in each set of wells is converted to a percentage of dye present in untreated control wells using a Microsoft Excel computer-based spreadsheet and normalized based on the virus control. The 50% effective (EC50, virus-inhibitory) concentrations and 50% cytotoxic (CC50, cell-inhibitory) concentrations are then calculated by regression analysis. The quotient of CC50 divided by EC50 gives the selectivity index (SI) value. Compounds showing SI values>10 are considered active.

Reduction of Virus Yield (Secondary VYR Assay)

Active compounds are further tested in a confirmatory assay. This assay is set up similar to the methodology described above only eight half-log 10 concentrations of inhibitor are tested for antiviral activity and cytotoxicity. After sufficient virus replication occurs (3 days for SARS-CoV-2), a sample of supernatant is taken from each infected well (three replicate wells are pooled) and tested immediately or held frozen at −80° C. for later virus titer determination. After maximum CPE is observed, the viable plates are stained with neutral red dye. The incorporated dye content is quantified as described above to generate the EC50 and CC50 values.

The VYR test is a direct determination of how much the test compound inhibits virus replication. Virus yielded in the presence of test compound is titrated and compared to virus titers from the untreated virus controls. Titration of the viral samples (collected as described in the paragraph above) is performed by endpoint dilution (Reed, L. J., and H. Muench. “A Simple Method of Estimating Fifty Percent Endpoints.” Am J Hyg 27 (1938): 493-98.)

Serial 1/10 dilutions of virus are made and plated into 4 replicate wells containing fresh cell monolayers of Vero 76 cells. Plates are then incubated, and cells are scored for the presence or absence of virus after a distinct CPE is observed, and the CCID50 calculated using the Reed-Muench method. The 90% (one log 10) effective concentration (EC90) is calculated by regression analysis by plotting the log 10 of the inhibitor concentration versus log 10 of virus produced at each concentration. Dividing EC90 by the CC50 gives the SI value for this test.

### Example 9: Results of Secondary VYR Assay

| | |
|---|---|
| NR | 29081 |
| ARB Number | 20-000260 |
| Drug Name | RBT-9 |
| Sponsor Company | Renibus Therapeutics, Inc |
| Date Received | Jul. 27, 2020 |
| Compound Class Type | Other Direct Antiviral |
| VEHICLE | DMSO |
| VIRUS | SARS-CoV-2 |
| VIRUS_ID | USA_WA1/2020 |
| Virus Code | SARS2 |
| Cell Line | Vero 76 |
| Drug Conc. (High) | 100 |
| Drug Conc. (Low) | 0.032 |
| Drug Units | μM |
| Control Conc. (High) | 100 |
| Control Conc. (Low) | 0.032 |
| Control Units | μg/ml |
| Start Date | Jul. 31, 2020 |
| Submitted by (PI) | Hurst |
| VIS EC50 | 1.5 |
| VIS CC50 | 27 |
| VIS SI | 18 |
| NR EC50 | 1.5 |
| NR CC50 | 39 |
| NR SI | 26 |
| CC50 for 2ary | 39 |
| TEST EC90 | 0.97 |
| SI BY VYR | 40 |
| POS VIS EC50 | 0.38 |
| POS VIS CC50 | 100 |
| POS VIS SI | 260 |
| POS NR EC50 | 0.32 |
| POS NR CC50 | 100 |
| POS NR SI | 310 |
| POS CC50 for 2ary | 100 |
| Positive EC90 | 0.2 |
| Positive SI by VYR | 500 |
| POS_ARB | 03-006467 |
| POS_NAME | M128533 |
| Exp_No | SARS2-210 |
| Primary, Secondary, Tertiary | S |
| Task Order Number | B02 |
| COMMENTS | 1 hour pretreatment of |
| | compound and virus |

The data above were obtained according to the Secondary VYR assay described in Example 8 above. Results from the VYR assay confirmed antiviral activity of RBT-9 against SARS-CoV-2. RBT-9 inhibited 90% of viral replication at a concentration of 0.97 μM (EC50). This concentration was 40 times lower than the 50% cytotoxic concentration of 39 μM.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

1. A method for treating an enveloped virus infection comprising:
administering a metal protoporphyrin or metal mesoporphyrin to a human patient at risk for developing complications from the enveloped virus infection.

2. The method of claim 1, wherein the metal protoporphyrin or metal mesoporphyrin is stannous protoporphyrin ix (SnPP), stannous mesoporphyrin ix, cobalt protoporphyrin ix, cobalt mesoporphyrin ix, zinc protoporphyrin ix, or zinc mesoporphyrin ix.

3. The method of claim 1, wherein the enveloped virus infection is SARS-COV-2.

4. The method of claim 1, wherein the patient has tested positive for a respiratory viral infection.

5. The method of claim 1, wherein the patient has tested positive for the respiratory viral infection and has not developed symptoms of pneumonia.

6. The method of claim 1, wherein the patient has been exposed to a SARS-COV-2 infection but has not tested positive for a coronavirus infection.

7. The method of claim 1, wherein the patient exhibits one or more risk factors for complication associated with SARS-COV-2.

8. The method of claim 1, wherein the patient is age 60 or above, has a d-dimer greater than 1 μg/L, has a structural organ failure assessment (SOFA) score of 1 or above.

9. The method of claim 1, wherein the patient is suffering from a comorbidity associated with greater risk for complication associated with SARS-COV-2.

10. The method of claim 1, wherein the patient is suffering from a comorbidity comprising one or more of hypertension, diabetes, coronary heart disease, or chronic obstructive lung disease.

11. A method for treating a coronavirus infection comprising:
administering a cobalamin to a human patient at risk for developing complications from coronavirus infection.

12. The method of claim 11, wherein the cobalamin is a cyanocobalamin, hydroxocobalamin, methylcobalamin, adenosylcobalamin, or 5-deoxyadenosyl cobalamin.

13. The method of claim 1, wherein the coronavirus is SARS-COV-2.

14. The method of claim 1, wherein the patient has tested positive for the coronavirus infection.

15. The method of claim 1, wherein the patient has tested positive for the coronavirus infections and has not developed symptoms of pneumonia.

16-20. (canceled)

21. A method of treating or preventing an envelope virus infection comprising administering an intranasal composition comprising a metal protoporphyrin or metal mesoporphyrin to a patient in need thereof.

22-29. (canceled)

30. A method of treating or preventing an enveloped virus infection comprising applying a face mask to a person, the facemask comprising an antiviral composition comprising a metal protoporphyrin or metal mesoporphyrin, wherein upon applying the facemask the metal protoporphyrin or metal mesoporphyrin enters the nasal cavity of the person in an amount effective to treat or prevent the enveloped virus infection.

31-35. (canceled)

36. An intranasal composition for the treatment or prevention of an enveloped virus infection, the intranasal composition comprising a metal protoporphyrin or metal mesoporphyrin, wherein the intranasal composition is suitable for human intranasal administration.

37-41. (canceled)

42. A method the reduction or prevention of spread of an enveloped virus comprising applying a face mask to a person, the facemask comprising an antiviral composition comprising a metal protoporphyrin or metal mesoporphyrin, wherein particles of the enveloped virus exhaled or expelled by the person into the facemask are destroyed or neutralized by the antiviral composition.

43-47. (canceled)
